(19) United States
(12) Patent Application Publication
(10) Pub. No.: US 2021/0290828 A1
(43) Pub. Date: Sep. 23, 2021

(54) Formation Of Therapeutic Scar Using Small Particles

(71) Applicants: Zhihao Yang, Vestal, NY (US); Richard Cornelius, Wayzata, MN (US); Paige Hastings, Bloomington, MN (US); Bodo Quint, Rottenburg (DE); Gerd Seibold, Ammerbuch (DE); Ib Erling Joergensen, Haigerloch (DE); Stevan Nielsen, Rottenburg Am Neckar (DE)

(72) Inventors: Zhihao Yang, Vestal, NY (US); Richard Cornelius, Wayzata, MN (US); Paige Hastings, Bloomington, MN (US); Bodo Quint, Rottenburg (DE); Gerd Seibold, Ammerbuch (DE); Ib Erling Joergensen, Haigerloch (DE); Stevan Nielsen, Rottenburg Am Neckar (DE)

(73) Assignee: SYNTACH AG, Schaffhausen (CH)

(21) Appl. No.: 12/298,287

(22) PCT Filed: May 8, 2007
(86) PCT No.: PCT/EP2007/054450

§ 371 Date: Jul. 27, 2012

Related U.S. Application Data
(60) Provisional application No. 60/799,122, filed on May 9, 2006.

(51) Int. Cl. A61L 31/10; A61L 27/30; A61L 31/08; A61L 31/14; A61L 27/54; A61L 27/44; A61L 27/58

(52) U.S. Cl. CPC A61L 31/10; A61L 27/303; A61L 31/088; A61L 31/148; A61L 27/54; A61L 27/44; A61L 2300/602; A61L 2400/12; A61L 2430/34; A61L 2300/622; A61L 2300/624; A61L 2300/102; A61L 2400/16; A61L 27/58

(57) ABSTRACT

The present invention generally relates to the use of small particles, such as micro particles or nanoparticles, to produce a therapeutic scar such as “trans-mural” scarring or other desired “deep tissue” scarring. In one preferred embodiment, these particles can be delivered to a target location by an implant. More specifically, these particles can be incorporated into the structure of implants or into the coatings on implants. In another preferred embodiment, these small particles can be delivered directly with a catheter by electrophoresis or hydraulic pressure.

### RELATED APPLICATIONS

This application claims priority to U.S. Provisional Application Ser. No. 60/799,122 filed May 9, 2006 entitled Microparticle Coating and to International Patent Application No. PCT/EP2007/054450 entitled Formation Of Therapeutic Scar Using Small Particles filed May 8, 2007, both of which are hereby incorporated by reference.

### BACKGROUND OF THE INVENTION

This application generally relates to techniques for scarring a desired tissue location in the human body. These techniques can be implemented through devices such as those described in U.S. Pat. No. 7,097,643 filed Mar. 2, 2004; and U.S. Publication Nos. 2004-0220655 filed Mar. 2, 2004; 2006-0116666 filed Oct. 7, 2005; and 2004-0254597 filed Apr. 30, 2004, each of which is incorporated by reference in its entirety, and in other possible delivery modes as described in this specification.

The previously noted applications describe devices that create clinically beneficial scarring at target locations in the human body. For example, this scar generation may be used for treatment of heart arrhythmias such as atrial fibrillation or treatment of certain cancers. In a more specific example, one such device creates scar tissue around the pulmonary vein ostium for treatment of atrial fibrillation. This ring of scar tissue stops transmission of electrical potentials from the pulmonary vein to the atrial tissue, thereby reducing or stopping arrhythmia.

The prior art describes different mechanisms for producing therapeutic scarring, such as mechanical pressure, energy ablation, inflammatory materials, ablative drugs and combinations thereof.

In some clinical applications, such as for electrical isolation of the pulmonary veins, it is important for the scar to extend through the full thickness of the tissue wall (“trans-mural” scarring) to yield the desired electrical isolation. However, prior art mechanisms may not achieve this trans-mural or deep tissue scarring necessary for some procedures (e.g., arrhythmia treatments). Hence, an improved method or apparatus for achieving such scarring would be advantageous.

### SUMMARY

Accordingly, embodiments of the present invention preferably seek to mitigate, alleviate or eliminate one or more deficiencies, disadvantages or issues in the art, such as the above-identified, singly or in any combination by providing a method and apparatus according to the appended patent claims.

One aspect of the present invention describes the use of small particles, such as micro particles or nanoparticles, to produce a therapeutic scar such as “trans-mural” scarring or other desired “deep tissue” scarring. In an embodiment, these particles may be delivered to a target location by an implant. More specifically, these particles may be incorporated into the structure of implants or into the coatings on implants. In other preferred embodiments, these particles may directly be delivered by electrophoresis or hydraulic injection into the target tissue.

In an aspect of the invention, a method of generating a substantially transmural scar in mammalian tissue is provided. The method comprises providing a plurality of particles, the particles sized for causing an inflammatory response in the mammalian tissue; introducing the particles into the mammalian tissue; causing the particles to disburse substantially into a thickness of the mammalian tissue; allowing the particles to inflame the tissue until a substantially transmural scar is generated in the mammalian tissue.

In a further aspect of the invention, an apparatus for causing a substantially transmural scar in body tissue is provided. The apparatus comprises a plurality of particles; a delivery device for introducing said particles to said body tissue; a mechanism for disbursing said particles substantially through a thickness of said body tissue; wherein said particles are sufficiently sized to cause a substantially transmural scar in said body tissue.

Further embodiments of the invention are defined in the dependent claims, wherein features for the different aspects of the invention apply mutatis mutandis.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates a side view of a preferred embodiment of an implant according to a preferred embodiment of the present invention;

FIG. 2A illustrates a end view of a preferred embodiment of an implant within an ostium of a pulmonary vein according to the present invention; and

FIG. 2B illustrates an enlarged view of the implant of FIG. 2A.

### DETAILED DESCRIPTION OF THE INVENTION

Specific embodiments of the invention now will be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention.

One aspect of this invention relates to how the body handles different sizes of foreign materials. In general, the body can handle perceived foreign materials in two ways. If the foreign material is relatively large, the body will respond by attempting to isolate the foreign material from the body by encasing it in scar tissue. This occurs with an inflammatory response around the surface of the foreign material which heals into an encasing layer of scar. If the foreign material is smaller, for example on the scale of a cell, the body will respond by trying to absorb and digest or “eat” the foreign material, as it does with virus.

This latter response may be used for treatment purposes by introducing materials which are at or below the size of cells, such as microparticles or nanoparticles. When delivered to a desired target location, for example by way of implants or coatings on implants, these particles are believed to promote scarring through macrophage/phagocytosis activation, often causing cell death of macrophages that attempt “digestion”. The death of these macrophage cells may result in inflammation deeper in the wall of the tissue from where the foreign material was originally deposited. Further, the inflammatory response is not limited to the identified foreign body, but also to the surrounding tissue. Phagocytosis and fibrosis (scarring) are concurrent inflammatory response which cause a deeper tissue scarring effect. Additionally, prior to death, the macrophage cell may transport the foreign material deeper into the tissue and thereby further increase the depth of the inflammatory response. The end result of this reaction is deeper and more aggressive scar generation which may be synergistically used with other scarring techniques (e.g., tissue cutting).

It should be also noted that in addition to size, other factors may influence the biological response, such as morphology, surface charge and area. With regard to particle morphology, nanofibers such as asbestos or carbon nanotubes are known to cause intensive and potential uncontrolled inflammatory response. They offer a very high surface area which is believed to be the cause for the identification as a foreign substance. Further, due to agglomeration, these materials can be difficult to be cleared by phagocytosis. Clay-like materials can offer the beneficial aspect ratio of fiber-like substances but due to their natural surface charge they separate in aqueous solutions and can be cleared by phagocytosis. This kind of additive is beneficial in order to create a controlled inflammatory response. In general, particle sizes greater than 1 μm can be difficult to be cleared by phagocytosis and in this example, signal substances are transmitted from phagocytes in order to stimulate more intensive fibrosis (scar generation) and phagocytosis.

With regard to surface properties, the amount of adsorption that is often key for the foreign body identification is dependent, at least in part, on surface area, surface charge and dispersive surface effects. In general, positive charged surfaces are less biocompatible due to effective adsorption of negatively charged body fluid contents. Most metal oxides and ceramic surfaces offer a more biocompatible surface due to their natural negative surface charge. Materials with a very low surface polarity and charge, such as PE and PTFE which are known to be highly biocompatible.

A further example factor that may influence the biological response is radiation. It is well known that radiation can directly cause cell death/necrosis. Therefore the use of radioactive particles may lead to long term identification as a foreign body.

Yet another example factor that may influence the biological response of the body is the use of enzymes, either as “foreign” particles or in addition to particles. During an inflammatory reaction, the body requires increased amounts of fibrin, plasmin, thrombin and kinins. However, the primary response to inflammation is through the immune system and its increased use of enzymes in order to support these raw materials. Localized deficiencies of enzymes can prolong inflammation and delay healing. Oral use of proteolytic enzymes may reduce healing times by up to 50 percent. In addition to proteases, amylases and lipases also play a role in the inflammatory response. Each contributes in specific functions to assist the inflammatory responses.

These enzymes can be gathered from several sources and concentrated for supplemental use. The use of fungal enzymes typically function in a broader pH range than those taken from beef and pork (so-called pancreatin). An important consideration may be that there are specific pH changes in tissue during inflammation so that the affected tissue comes either more alkaline or more acidic than normal. Plant enzymes have a broader range of substrates that can be used. Further, digestive enzymes from the pancrease (e.g., collagenases and proteases) are selective in the bonds they can hydrolyze and therefore digest.

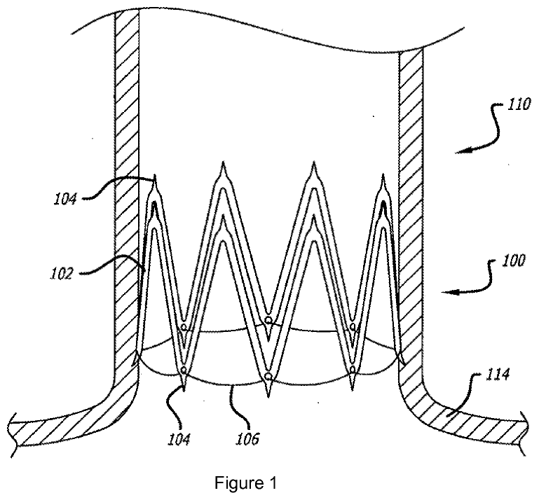

FIG. 1 illustrates an embodiment of a self expanding mechanical implant 100 according to the present invention located near an ostium 114 of a pulmonary vein 110. The implant includes a plurality of struts 102 that are configured to exert a mechanical pressure against the desired target tissue. The peaks where each strut 102 connects to the next strut 102 includes an anchoring barb 104 which is shaped to pierce the target tissue and therefore provide anchoring support to the implant 100. The implant 100 optionally includes a wire 106 that is fixed to the peak of the struts 102 on one end of the implant 100 for creating a narrow band of pressure on the target tissue. Further details of the self expanding mechanical implant 100, and similar Nitinol implants, can be seen in U.S. Publication No. 2004-0220655 filed Mar. 2, 2004 and U.S. Publication No. 2006-0116666 filed Oct. 7, 2005 (previously incorporated by reference).

The implant 100 is further coated with a biodegradable polymer such as 50/50 PLGA loaded with 50% by weight of 2 micron copper particles. Preferably, the 50/50 PLGA will have largely or completely degraded after 30 days thereby freeing the 2 micron particles to direct exposure to the tissue. After 30 days there will be significantly more inflammation around the implant than would be possible with no coating, only a PLGA coating or even only a solid copper plating. Further, this inflammation caused by the implant 100 will extend much deeper into the tissue wall than for copper plated devices.

With additional time, inflammation from the implant 100 resolves into a scar yielding the desired deeper scarring from the deeper inflammation generated by the small particle copper loaded coating. It is believed that the body's digestive immune response (phagocytosis) to the foreign material occurs with particles that are as large as 10-20 microns and be fully evident as the particle size reduces to 5 micron or less. Further, an inflammation response is also possible with the use of even smaller particles such as nanoparticles. Additional information about nanoparticles can be found in Nanotechnology: A Brief Literature Review by M. Ellin Doyle, Ph.D., June 2006 University of Wisconsin FRI Briefings and in Industrial Application of Nanomaterials—Chances and Risks, Wolfgang Luther (ed.), VDI Technologiezentrum GmbH, the contents of both references incorporated herein by reference in their entirety.

This previously described principal (i.e., a bodily immune response to small particles) may according to certain embodiments be applied to other materials as well (e.g., materials with other mechanisms of causing inflammation). Moreover, the total amount of the inflammatory particles introduced may be varied through control of the coating thickness or the percent weight loading of the particles in coating. In this respect, the thickness of scar generation may be adjusted to achieve a desired result.

Further, the inflammatory particles may be incorporated to be released in predetermined quantities over a period of time. For example, inflammatory materials may be filled, coumpounded or otherwise incorporated into additional materials that degrade within the body after predetermined times, thereby delaying inflammatory reactions after implantation. In another embodiment, such time release techniques may be used to release constant amounts of inflammatory materials over an extended period of time or provide multiple “spikes” of inflammatory material at predetermined times after delivery of a device within a patient.

In another embodiment, the scar generating material may be a drug such as Actinomycin, FUDR or Vinblastin as examples. These drugs can be encapsulated in biodegradable micro-spheres having diameters of less than about 5 microns made of materials such as, but not limited to, PLGA, PLA, Polyanhydride, or chitosan. The micro-sphere material may also be made of a durable material which allows diffusion of the encapsulated drug. These micro-spheres may then be coated directly to the surface of an implant or adhered to the implant with a binder material.

The construction of this embodiment allows the micro-spheres to diffuse or to be carried by macrophages deeper into the tissue before delivering the encapsulated drug or material. Thus, this technique may achieve deeper scarring as compared to direct delivery. Further, the encapsulated drug itself (i.e., not just the size of the drug particle) may create a more intense inflammatory response and therefore create more dense scar tissue.

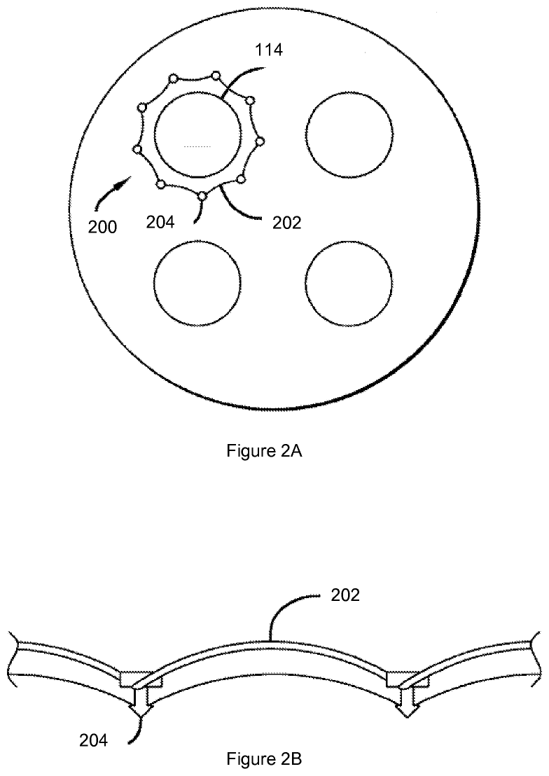

FIGS. 2A and 2B illustrate another embodiment of an implant 200 that delivers inflammatory particles via a strand 202 made with a biodegradable polymer matrix. The strand 202 is connected to a plurality of staples 204. The staples 204 are punctured into the tissue, fastening the strand 202 against the target tissue. Further details of this implant and example delivery systems can be seen in U.S. Pat. No. 7,097,643 filed Mar. 2, 2004, (e.g. FIGS. 21a and 21b) and U.S. Publication No. 2004-0220655, filed Mar. 2, 2004 FIG. 21 (both incorporated by reference). The delivery device feeds out lengths of the particle loaded strand 202 with staples 204 attached at intervals along the length (e.g., every 5 millimeters).

In this way, the steerable delivery catheter may be maneuvered over the desired path of scar generation, leaving behind a line of the particle containing strand 202. This path may be visualized during the procedure using either radioopaque markers incorporated into the staples 204 or radioopaque loading of the strand material.

In another embodiment, the staples of the previously described embodiment are formed of a superelastic and/or shape memory material, such as a polymer or metal, for instance Nitinol, in an open ring shape with the ends being in close proximity to each other (e.g., a “C” shape). As the staple feeds out the tip of a delivery catheter, the ends of the staples are flexed apart and are pressed against the tissue by the tip of the catheter. In this way, when the staple is released, the ends spring back together and embed in the tissue at the targeted site. This anchors the strand to the tissue at this point. Alternatively, another mechanism of adhering the strand to the tissue surface is a bio-adhesive.

In additional preferred embodiments, inflammatory particles (e.g., microparticles or nanoparticles) may be released from materials such as filled degradable polymers, crystallites of degradable semi crystalline plastics, contents of plastic blends, copolymers, fiber containing materials, and carbon fibers. Further, instead of a matrix based on degradable polymers, degradable ceramics or degradable metals may be used.

In another embodiment, the particles may be delivered directly into the tissue at the targeted site without any additional implant using direct injection of the particles. This may be performed using the technique of electrophoresis on a charged particles surface or particle coating to enable the electrophoreretic delivery. An electrode at the end of the catheter drives the particles away from the catheter tip and into the tissue when the electrode is polarized. This basic technology is well known and examples are shown in U.S. Pat. Nos. 4,411,648; 5,807,306; 5,704,908; and 6,219,577, each of which is herein incorporated by reference in their entirety.

The direct delivery of the particles (e.g., microparticles or nanoparticles) into the tissue may also be accomplished using hydraulic pressure for particle injection. This technique is also a commonly known method, whereby the particles are injected using fluid pressure either through micro-needle(s) or nozzle(s) at the tip of the catheter or around the circumference of a centering device, for example placed in the ostium of the pulmonary vein. Examples of devices to enable this type of delivery are shown in U.S. Pat. Nos. 5,306,250; 5,538,504; or 6,254,573, each of which is incorporated by reference in their entirety.

In another embodiment, carbon fibers may be used to cause inflammation and enhance the modulus of composites (e.g., by enhancing the forces provided by a mechanical implant). The carbon fibers may be incorporated into a resorbable matrix, thereby offering a high surface area that can be identified as a foreign body. The inflammatory reaction of carbon fibers has been described in some literature as “low and not critical”. However, using, for example, textile fiber finishing methods or plasma treatment, the surface charge and polarity may be modified. Thus, due to their mechanical reinforcement and low inflammatory reaction, carbon fibers may synergistically compliment the effects of traditional scar generating techniques (e.g., cutting or pressure).

Although the invention is described herein with respect to specific embodiments, the scope of the invention is in no way limited thereby. It is understood that one of ordinary skill in the art can contemplate variations and improvements to the ideas presented herein without departing from the scope of the claimed invention as defined in the appended patent claims.

1. An apparatus adapted to cause a desired substantially transmural scar in a body tissue, said apparatus comprising:
a plurality of particles;
a delivery device configured to introduce said particles into said body tissue;
a mechanism for disbursing said particles substantially through a thickness of said body tissue;
said particles being sufficiently sized to cause said substantially transmural scar in said body tissue.

2. The apparatus of claim 1, wherein said particles are inflammatory particles, and wherein a total amount of said inflammatory particles is controlled by a thickness of a coating of said delivery device or percent weight loading of the inflammatory particles in said coating, such that a thickness of scar generation is target adjustable.

3. The apparatus of claim 1, wherein said particles are inflammatory particles that are incorporated in said delivery device to be released in predetermined quantities over a period of time.

4. The apparatus of claim 3, wherein said inflammatory particles are incorporated into additional materials that degrade within the body after predetermined times, such that inflammatory reactions are delayed after delivery.

5. The apparatus of claim 3, comprising time release mechanisms in use of said device releasing constant amounts of inflammatory particles over an extended period of time or providing multiple spikes of inflammatory particles at predetermined times after delivery of said delivery device within a patient.

6. The apparatus of claim 1, wherein said particles comprise a drug encapsulated in biodegradable micro-spheres having diameters of less than about 5 microns, and wherein said particles are made of PLGA, PLA, Polyanhydride, or chitosan.

7. The apparatus of claim 6, wherein said micro-sphere material are made of a durable material which allows diffusion of the encapsulated drug.

8. The apparatus of claim 7, wherein said micro-spheres are coated to the surface of an implant or adhered to said implant with a binder material.

9. The apparatus of claim 1, wherein said delivery device is an implant (200) arranged to said particles via a strand (202) made with a biodegradable polymer matrix, wherein said strand (202) is connected to a plurality of staples (204) arranged to be punctured into said body tissue, in use of the apparatus fastening the strand (202) against said body tissue.

10. The apparatus of claim 9, wherein said staples (204) comprise radioopaque markers, or said strand (202) comprises a material having a radioopaque loading.

11. The apparatus of claim 9, wherein said staples are formed of a superelastic material.

12. The apparatus of claim 1, wherein said particles are inflammatory particles, arranged in filled degradable polymers, crystallites of degradable semi crystalline plastics, contents of plastic blends, copolymers, fiber containing materials, and carbon fibers.

13. The apparatus of claim 1, wherein said particles are inflammatory particles, arranged in degradable ceramics or degradable metals.

14. The apparatus of claim 1, wherein said delivery device comprises an implant, or a self expanding mechanical implant.

15. The apparatus of claim 14, wherein said implant further comprises a coating, said coating containing said particles.

16. The apparatus of claim 15, wherein said coating is arranged to degrade within a body over a predetermined time.

17. The apparatus of claim 14, wherein said implant contains said particles.

18. The apparatus of claim 1, wherein said particles are copper.

19. The apparatus of claim 1, wherein said particles are fiber, or carbon fiber.

20. The apparatus of claim 1, wherein said particles are encapsulated in micro-spheres.

21. The apparatus of claim 1, wherein said delivery device is an electrophoresis delivery device.

22. The apparatus of claim 1, wherein said delivery device is a hydraulic delivery device.

23. The apparatus of claim 1, wherein said particles are less than 20 microns in size.

24. The apparatus of claim 1, wherein said particles are less than 5 microns in size.

25. The apparatus of claim 1, wherein said particles are nanoparticles.

26. A method of generating a substantially transmural scar in mammalian tissue comprising:
providing a plurality of particles, said particles sized for causing an inflammatory response in said mammalian tissue;
introducing said particles into said mammalian tissue;
causing said particles to disburse substantially into a thickness of said mammalian tissue;
allowing said particles to inflame said tissue until a substantially transmural scar is generated in said mammalian tissue.

27. The method of claim 26, wherein said particles are less than 20 microns in size.

28. The method of claim 26, wherein said particles are less than 5 microns in size.

29. The method of claim 26, wherein said particles are nanoparticles.

30. The method of claim 26, wherein said introducing said particles into mammalian tissue further comprises implanting an implant at a target location within a mammalian body.

31. The method of claim 30, wherein said causing said particles to disburse substantially into a thickness of said mammalian tissue further comprises releasing said particles from said implant.

32. The method of claim 30, wherein said causing said particles to disburse substantially into a thickness of said mammalian tissue further comprises releasing said from a coating on said implant.

33. The method of claim 26, wherein said introducing said particles into mammalian tissue further comprises delivering said particles in micro-spheres.

34. The method of claim 26, wherein said introducing said particles into mammalian tissue further comprises applying a force on a charged coating on said particles with electrophoresis.

35. The method of claim 26, wherein said introducing said particles into mammalian tissue further comprises applying a hydraulic pressure on said particles.
